(19) United States
(12) Patent Application Publication
(10) Pub. No.: US 2025/0009737 A1
(43) Pub. Date: Jan. 9, 2025

(54) COMPOUNDS AND METHODS FOR THE TARGETED DEGRADATION OF ANDROGEN RECEPTOR

(71) Applicant: Arvinas Operations, Inc., New Haven, CT (US)

(72) Inventors: Jingang George ZHANG, North Andover, MA (US); Lawrence B. SNYDER, Killingworth, CT (US); Yongqing HUANG, Flemington, NJ (US)

(21) Appl. No.: 18/759,774

(22) Filed: Jun. 28, 2024

Related U.S. Application Data
(60) Provisional application No. 63/511,420, filed on Jun. 30, 2023.
(60) Provisional application No. 63/580,126, filed on Sep. 1, 2023.

(51) Int. Cl. A61K 31/496; A61K 45/06

(52) U.S. Cl. CPC A61K 31/496; A61K 45/06

(57) ABSTRACT

This disclosure pertains to methods of treating prostate cancer in a subject in need thereof, including, for example, metastatic prostate cancer, castrate-resistant prostate cancer, and metastatic castrate-resistant prostate cancer, wherein the method comprises administering to the subject a therapeutically effective amount of Compound A, or a pharmaceutically acceptable salt thereof, and further comprises a step of discontinuing or reducing the administration of a CYP3A inhibitor or inducer, an efflux transporter substrate or inhibitor, or an uptake transporter substrate or inhibitor, to the subject prior to initiating administration of Compound A

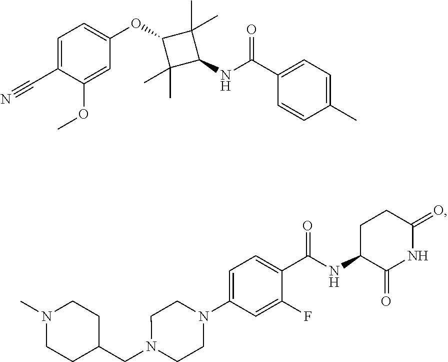

or the pharmaceutically acceptable salt thereof.

### RELATED APPLICATIONS

This application claims priority to, and the benefit of, U.S. Provisional Application No. 63/511,420, filed Jun. 30, 2023, and U.S. Provisional Application No. 63/580,126, filed Sep. 1, 2023, the contents of each of which are incorporated herein by reference in their entirety for all purposes.

### TECHNICAL FIELD

The disclosure provides methods of using bifunctional compounds for treating prostate cancer.

### BACKGROUND OF THE DISCLOSURE

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and are therefore attractive therapeutic targets. The development of ligands of E3 ligases has proven challenging, in part due to the fact that they must disrupt protein-protein interactions. However, recent developments have provided specific ligands which bind to these ligases.

One E3 ubiquitin ligase with therapeutic potential is cereblon. Cereblon is a protein that in humans is encoded by the CRBN gene. Thalidomide and its analogs, e.g., pomalidomide and lenalidomide, are known to bind cereblon. These agents bind to cereblon, altering the specificity of the complex to induce the ubiquitination and degradation of transcription factors essential for multiple myeloma growth. Indeed, higher expression of cereblon has been linked to an increase in efficacy of imide drugs in the treatment of multiple myeloma.

Androgen Receptor (AR) belongs to a nuclear hormone receptor family that is activated by androgens, such as testosterone and dihydrotestosterone (Pharmacol. Rev. 2006, 58(4), 782-97; Vitam. Horn. 1999, 55:309-52). In the absence of androgens, AR is bound by Heat Shock Protein 90 (Hsp90) in the cytosol. When an androgen binds AR, its conformation changes to release AR from Hsp90 and to expose the Nuclear Localization Signal (NLS). The latter enables AR to translocate into the nucleus where AR acts as a transcription factor to promote gene expression responsible for male sexual characteristics (Endocr. Rev. 1987, 8(1):1-28; Mol. Endocrinol. 2002, 16(10), 2181-7). AR deficiency leads to Androgen Insensitivity Syndrome, formerly termed testicular feminization.

While AR is responsible for development of male sexual characteristics, it is also a well-documented oncogene in certain forms of cancers including prostate cancers (Endocr. Rev. 2004, 25(2), 276-308). A commonly measured target gene of AR activity is the secreted Prostate Specific Antigen (PSA) protein. The current treatment regimen for prostate cancer involves inhibiting the androgen-AR axis by two methods. The first approach relies on reduction of androgens, while the second strategy aims to inhibit AR function (Nat. Rev. Drug Discovery, 2013, 12, 823-824). Despite the development of effective targeted therapies, most patients develop resistance and the disease progresses. An alternative approach for the treatment of prostate cancer involves eliminating the AR protein.

Because AR is a critical driver of tumorigenesis in many forms of prostate cancers, its elimination should lead to a therapeutically beneficial response. There exists an ongoing need in the art for effective treatments for diseases, especially cancer, prostate cancer, and Kennedy's Disease.

However, non-specific effects, and the inability to target and modulate certain classes of proteins altogether, such as transcription factors, remain as obstacles to the development of effective anti-cancer agents. As such, small molecule therapeutic agents that leverage or potentiate cereblon's substrate specificity and, at the same time, are “tunable” such that a wide range of protein classes can be targeted and modulated with specificity would be very useful as a therapeutic.

Over 70 different somatic missense AR tumor mutation have been identified in patients with prostate cancer (Gottlieb, B., Hum. Mutat. 2004, 23:527-533). The majority of these AR tumor mutations reside in the ligand binding domain. Without being bound by theory, AR tumor mutations in the ligand binding domain result in decreased ligand specificity, thereby enabling AR to function independently of androgen. Such AR tumor mutations provide tumor cells with the capability to proliferate in androgen-depleted environments, and thus are selected in response to therapies for prostate cancer that block or reduce androgen levels (e.g., luteinizing hormone-releasing hormone agonists). Accordingly, AR tumor mutations are observed with increased frequency in patients having advanced, androgen-independent tumors as compared to patients having early-stage prostate cancer (Taplin, M. E., et al. N. Engl. J. Med. (1995) 332:1393-1398; Marcelli, M., et al. Cancer Res. (2000) 60:944-949).

### SUMMARY

In one aspect, this application pertains to a method of treating prostate cancer in a subject in need thereof comprising administering to the subject a therapeutically effective amount of Compound A,

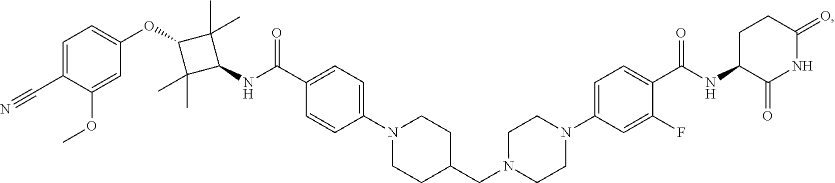 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, further comprising a step of discontinuing or reducing the administration of a CYP3A inhibitor to the subject prior to initiating administration of Compound A, or the pharmaceutically acceptable salt thereof.

In one aspect, this application pertains to a method of treating prostate cancer in a subject in need thereof comprising administering to the subject a therapeutically effective amount of Compound A,

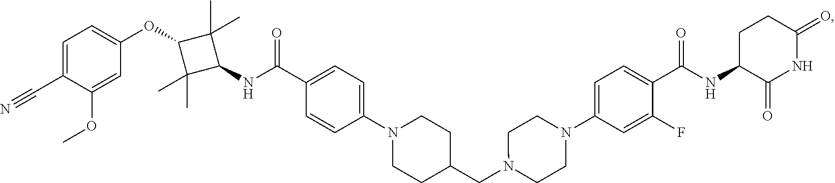 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

further comprising a step of discontinuing or reducing the administration of a CYP3A inhibitor to the subject prior to initiating administration of Compound A.

In one aspect, this application pertains to a method of treating prostate cancer in a subject in need thereof comprising administering to the subject a therapeutically effective amount of Compound A,

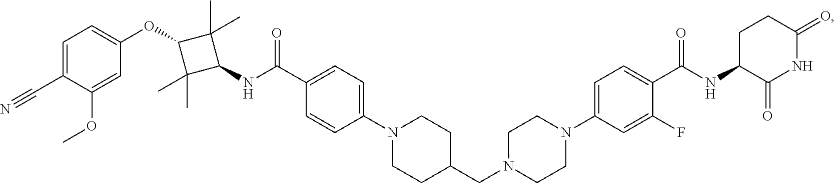 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, further comprising a step of discontinuing the administration of a CYP3A inhibitor to the subject prior to initiating administration of Compound A, or the pharmaceutically acceptable salt thereof. In some embodiments, the administration of the CYP3A inhibitor is discontinued in the subject beginning at a time point prior to initiating the administration of Compound A, or the pharmaceutically acceptable salt thereof, wherein the time point is at least 120 hours.

In one aspect, this application pertains to a method of treating prostate cancer in a subject in need thereof comprising administering to the subject a therapeutically effective amount of Compound A,

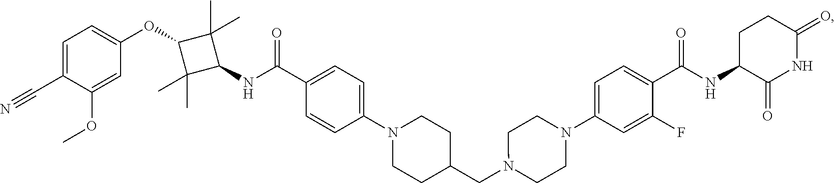 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

further comprising a step of discontinuing the administration of a CYP3A inhibitor to the subject prior to initiating administration of Compound A. In some embodiments, the administration of the CYP3A inhibitor is discontinued in the subject beginning at a time point prior to initiating the administration of Compound A, wherein the time point is at least 120 hours.

In one aspect, this application pertains to a method of treating prostate cancer in a subject in need thereof comprising administering to the subject a therapeutically effective amount of Compound A,

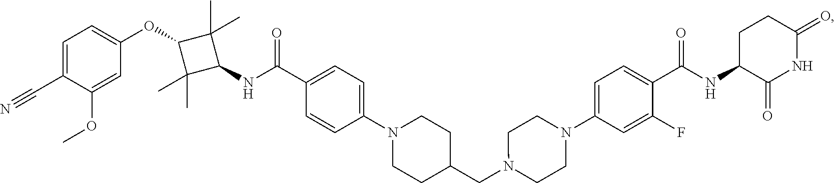 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, further comprising a step of reducing the administration of a CYP3A inhibitor to the subject prior to initiating administration of Compound A, or the pharmaceutically acceptable salt thereof. In some embodiments, the administration of the CYP3A inhibitor is reduced in the subject at a time point prior to initiating the administration of Compound A, or the pharmaceutically acceptable salt thereof, wherein the time point is at least 120 hours.

In one aspect, this application pertains to a method of treating prostate cancer in a subject in need thereof comprising administering to the subject a therapeutically effective amount of Compound A,

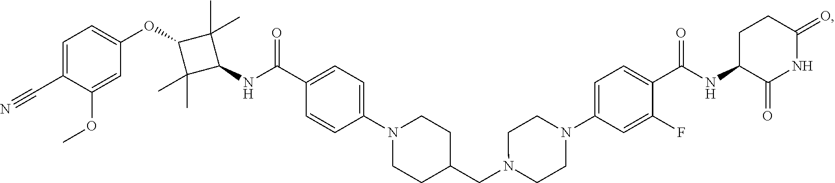 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

further comprising a step of reducing the administration of a CYP3A inhibitor to the subject prior to initiating administration of Compound A. In some embodiments, the administration of the CYP3A inhibitor is reduced in the subject at a time point prior to initiating the administration of Compound A, wherein the time point is at least 120 hours.

In one aspect, this application pertains to a method of treating prostate cancer in a subject in need thereof comprising administering to the subject a therapeutically effective amount of Compound A,

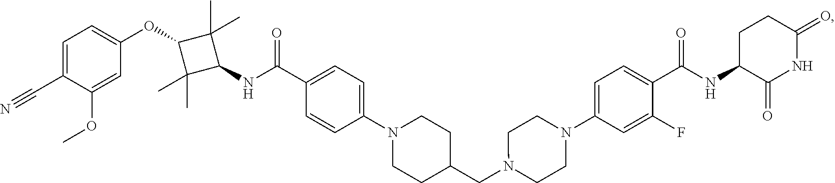 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, further comprising administering a CYP3A inhibitor to the subject.

In one aspect, this application pertains to a method of treating prostate cancer in a subject in need thereof comprising administering to the subject a therapeutically effective amount of Compound A,

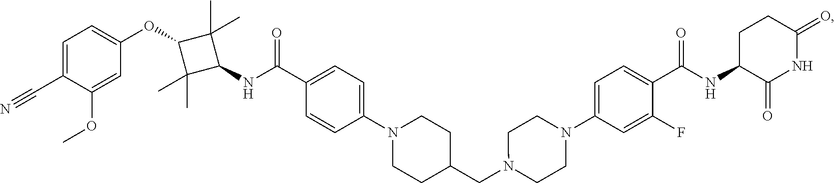 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

further comprising administering a CYP3A inhibitor to the subject.

In one aspect, this application pertains to a method of treating prostate cancer in a subject in need thereof comprising administering to the subject a therapeutically effective amount of Compound A,

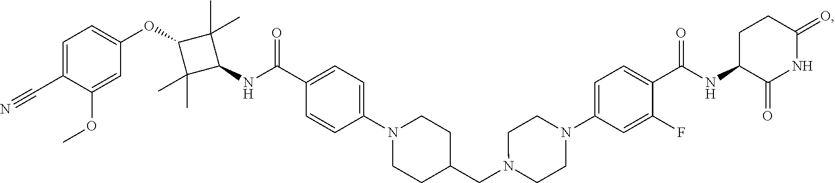 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, wherein the subject is also administered a CYP3A inhibitor.

In one aspect, this application pertains to a method of treating prostate cancer in a subject in need thereof comprising administering to the subject a therapeutically effective amount of Compound A,

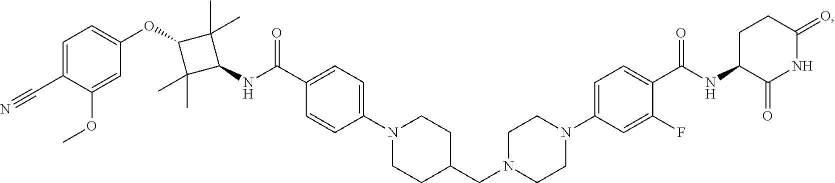 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the subject is also administered a CYP3A inhibitor.

In some embodiments, the therapeutically effective amount of Compound A, or the pharmaceutically acceptable salt thereof, is administered orally to the subject.

In some embodiments, the therapeutically effective amount of Compound A, or the pharmaceutically acceptable salt thereof, is administered to the subject once a day, twice a day, three times a day, or four times a day.

In some embodiments, the therapeutically effective amount of Compound A, or the pharmaceutically acceptable salt thereof, is administered to the subject once a day.

In some embodiments, the therapeutically effective amount of Compound A, or the pharmaceutically acceptable salt thereof, is administered to the subject all at once or is administered in two, three, or four divided doses.

In some embodiments, the therapeutically effective amount of Compound A is about 1 mg to about 1000 mg.

In some embodiments, the therapeutically effective amount of Compound A is about 5 mg to about 750 mg.

In some embodiments, the therapeutically effective amount of Compound A is about 10 mg to about 500 mg.

In some embodiments, the therapeutically effective amount of Compound A is about 20 mg to about 250 mg.

In some embodiments, the therapeutically effective amount of Compound A is about 100 mg.

In some embodiments, the therapeutically effective amount of Compound A is 100 mg.

In some embodiments, the therapeutically effective amount of Compound A is about 150 mg.

In some embodiments, the therapeutically effective amount of Compound A is 150 mg.

In some embodiments, the therapeutically effective amount of Compound A is about 300 mg.

In some embodiments, the therapeutically effective amount of Compound A is 300 mg.

In some embodiments, the method further comprises administering an effective amount of at least one additional anti-cancer agent to the subject in need thereof.

In some embodiments, the additional anti-cancer agent is abiraterone, abiraterone acetate, estramustine, docetaxel, ketoconazole, goserelin, histrelin, triptorelin, buserelin, cyproterone, flutamide, bicalutamide, nilutamide, pamidronate, or zolendronate.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIGS. 1A-F summarize the effect of Compound A on cytochrome P450 (CYP) CYP1A2 (FIG. 1A), CYP2B6 (FIG. 1B), CYP3A4 (FIG. 1C), CYP2C8 (FIG. 1D), CYP2C9 (FIG. 1E), and CYP2C19 (FIG. 1F) mRNA expression in human hepatocytes. Each grouped bar chart shows mRNA fold induction for 3 donors of human hepatocytes at varying concentrations of Compound A (0.03-30 UM).

FIGS. 2A-B summarize P-glycoprotein (Pgp, FIG. 2A) and breast cancer resistance protein (BCRP, FIG. 2B) inhibition by Compound A in MDCKII monolayer and vesicles expressing Pgp and BCRP. Each chart shows relative Pgp or BCRP activity (% of control) at varying concentrations of Compound A (μM) for vesicle and monolayer data. Data are the mean±standard deviation from triplicate samples.

### DETAILED DESCRIPTION

### Definitions

The term “Ubiquitin Ligase” refers to a family of proteins that facilitate the transfer of ubiquitin to a specific substrate protein, targeting the substrate protein for degradation. For example, cereblon is an E3 Ubiquitin Ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

“Pharmaceutically acceptable salt”, as used herein with respect to a compound of the disclosure, means a salt form of Compound A as well as hydrates of the salt form with one or more water molecules present. Such salt and hydrated forms retain the biological activity of Compound A and are not biologically or otherwise undesirable, i.e., exhibit minimal, if any, toxicological effects. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “isomer” refers to salts and/or compounds that have the same composition and molecular weight but differ in physical and/or chemical properties. The structural difference may be in constitution (geometric isomers) or in the ability to rotate the plane of polarized light (stereoisomers). With regard to stereoisomers, the salts of the compounds of the disclosure may have one or more asymmetric carbon atom and may occur as racemates, racemic mixtures and as individual enantiomers or diastereomers.

The compounds of the disclosure may exist in unsolvated as well as solvated forms such as, for example, hydrates.

“Solvate” means a solvent addition form that contains either a stoichiometric or non-stoichiometric amounts of solvent. Non-limiting examples of suitable solvates include ethanolate, methanolate, and the like. Some compounds have a tendency to trap a fixed molar ratio of solvent molecules in the crystalline solid state, thus forming a solvate. If the solvent is water, the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds of the disclosure.

“Isotopic derivative”, as referred to herein, relates to a compound of the disclosure that is isotopically enriched or labelled (with respect to one or more atoms of the compound) with one or more stable isotopes. Thus, in this application, the compounds of the disclosure include, for example, compounds that are isotopically enriched or labelled with one or more atoms such as deuterium.

As used herein, “treating” describes the management and care of a subject for the purpose of combating a disease, condition, or disorder and includes decreasing or alleviating the symptoms or complications, or eliminating the disease, condition or disorder.

The term “treating”, as used herein, unless otherwise indicated, means reversing, alleviating, or inhibiting the progress of the disorder or condition to which such term applies, or one or more symptoms of such disorder or condition. The term “treatment”, as used herein, unless otherwise indicated, refers to the act of “treating” as defined immediately above. For example, the terms “treat”, “treating” and “treatment” can refer to a method of alleviating or abrogating a particular disorder and/or one or more of its attendant symptoms.

As used herein, “subject” means a human or animal (in the case of an animal, the subject can be a mammal). In one aspect, the subject is a human. In one aspect, the subject is a male.

Prostate cancer is the uncontrolled growth of cancerous cells in the prostate gland.

Metastatic prostate cancer, or metastases, refers to prostate cancer that has spread beyond the prostate to other parts of the body, e.g., bones, lymph nodes, liver, lungs, brain.

Castrate-resistant prostate cancer or castration-resistant prostate cancer (or prostate cancer that is castrate- or castration-resistant) is a type of prostate cancer that keeps growing even when the amount of testosterone in the body is reduced to very low levels.

Metastatic castrate-resistant prostate cancer is a type of prostate cancer that has metastasized and continues to grow even when the amount of testosterone in the body is reduced to very low levels.

As used herein, “preventing” describes stopping the onset of the symptoms or complications of the disease, condition or disorder.

“Administration” refers to introducing an agent, such as a compound of the disclosure into a subject. The related terms “administering” and “administration of” (and grammatical equivalents) refer both to direct administration, which may be administration to a subject by a medical professional or by self-administration by the subject, and/or to indirect administration, which may be the act of prescribing a drug. For example, a physician who instructs a patient to self-administer a drug and/or provides a patient with a prescription for a drug is administering the drug to the patient.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are co-administered in combination with at least one additional bioactive agent, especially including an anti-cancer agent. In particularly preferred aspects, the co-administration of compounds results in synergistic activity and/or therapy, including anticancer activity.

“Therapeutically effective amount”, as used herein means an amount of the free base of Compound A, or the equivalent amount of a pharmaceutically acceptable salt of Compound A, that is sufficient to treat, ameliorate, or prevent a specified disease (e.g., prostate cancer), disease symptom, disorder or condition, or to exhibit a detectable therapeutic or inhibitory effect. The effect can be detected by any assay method known in the art. The effective amount for a particular subject may depend upon the subject's body weight, size, and health; the nature and extent of the condition; and whether additional therapeutics are to be administered to the subject. Therapeutically effective amounts for a given situation can be determined by routine experimentation that is within the skill and judgment of the clinician.

“Cₘₐₓ”, as used herein, refers to the observed maximum (peak) plasma concentration of a specified compound in the subject after administration of a dose of that compound to the subject.

“AUC”, as used herein, refers to the total area under the plasma concentration-time curve, which is a measure of exposure to a compound of interest, and is the integral of the concentration-time curve after a single dose or at steady state. AUC is expressed in units of ng*H/mL (ng×H/mL), where “H” refers to hours.

“AUCₜₐᵤ”, as used herein, refers to the AUC from 0 hours to the end of a dosing interval.

“AUC₀₋₂₄” means the AUC from 0 hours to 24 hours after administration of a single dose.

“Controlled release” or “CR” as used herein with respect to an oral dosage form refers to where a compound of the disclosure is released from the dosage form according to a predetermined profile that may include when and where release occurs after oral administration and/or a specified rate of release over a specified time period.

“Controlled release agent” as used herein with respect to an oral dosage form of the disclosure refers to one or more substances or materials that modulate release of a compound of the disclosure from the dosage form. Controlled release agents may be materials which are organic or inorganic, naturally occurring or synthetic, such as polymeric materials, triglycerides, derivatives of triglycerides, fatty acids and salts of fatty acids, talc, boric acid, colloidal silica, and combinations thereof.

“Enteric coating” as used herein with respect to a dosage form of the disclosure refers to a pH-dependent material that surrounds a core comprising a compound of the disclosure and which remains substantially intact in the acid environment of the stomach, but which dissolves in the pH environment of the intestines.

“Gastro-resistant” or “GR” as applied to a CR oral dosage form described herein means that release of a compound of the disclosure in the stomach of a subject shall not exceed 5%, 2.5%, 1% or 0.5% of the total amount of Compound A in the dosage form.

“Oral dosage form” as used herein refers to a pharmaceutical drug product that contains a specified amount (dose) of a compound of the disclosure as the active ingredient, or a pharmaceutically acceptable salt and/or solvate thereof, and inactive components (excipients), formulated into a particular configuration that is suitable for oral administration, such as an oral tablet, liquid, or capsule. In some embodiments, the compositions are in the form of a tablet that can be scored.

The term “carrier”, as used in this disclosure, encompasses pharmaceutically acceptable excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “about” as part of a quantitative expression such as “about X”, includes any value that is 10% higher or lower than X, and also includes any numerical value that falls between X−10% and X+10%. Thus, for example, a weight of about 40 g includes a weight of between 36 to 44 g.

Compound A of the present disclosure refers to 4-(4-((1-(4-(((1R,3R)-3-(4-cyano-3-methoxyphenoxy)-2,2,4,4-tetramethylcyclobutyl) carbamoyl)phenyl) piperidin-4-yl)methyl) piperazin-1-yl)-N—((S)-2,6-dioxopiperidin-3-yl)-2-fluorobenzamide, which has the following structure:

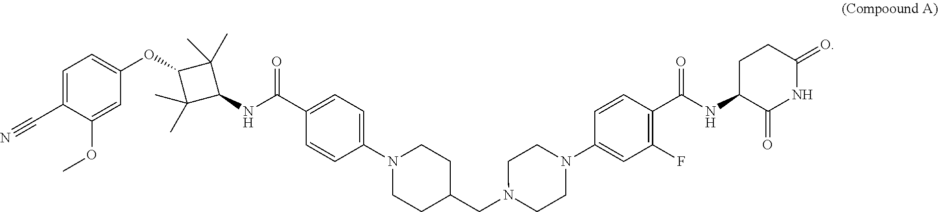 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Compound A can be prepared as described in US Patent Application Publication No. 2021/0196710 A1, which is incorporated herein by reference.

“Comprising” or “comprises” as applied to a particular dosage form, composition, use, method or process described or claimed herein means that the dosage form, composition, use, method, or process includes all of the recited elements in a specific description or claim, but does not exclude other elements. “Consists essentially of” and “consisting essentially of” means that the described or claimed composition, dosage form, method, use, or process does not exclude other materials or steps that do not materially affect the recited physical, pharmacological, pharmacokinetic properties or therapeutic effects of the composition, dosage form, method, use, or process. “Consists of” and “consisting of” means the exclusion of more than trace elements of other ingredients and substantial method or process steps.

The ECOG Scale of Performance Status was developed by the Eastern Cooperative Oncology Group as standard criteria for measuring how the disease impacts a patient's daily living abilities. It describes a patient's level of functioning in terms of their ability to care for themself, daily activity, and physical ability (walking, working, etc.). Patients are categorized on a scale of 1-5:

| GRADE | ECOG PERFORMANCE STATUS |
|---|---|
| 0 | Fully active, able to carry on all pre-disease performance without restriction |
| 1 | Restricted in physically strenuous activity but ambulatory and able to carry out |
| | work of a light or sedentary nature, e.g., light housework, office work |
| 2 | Ambulatory and capable of all selfcare but unable to carry out any work activities; |
| | up and about more than 50% of waking hours |
| 3 | Capable of only limited selfcare; confined to bed or chair more than 50% of waking |
| | hours |
| 4 | Completely disabled; cannot carry on any selfcare; totally confined to bed or chair |
| 5 | Dead |

A cytochrome P450 3A inhibitor, i.e., a “CYP3A inhibitor”, refers to a member of a class of compounds or a substance that reduces or inhibits the normal function of cytochrome P450 in humans. In some embodiments, the CYP3A inhibitor is a CYP3A4 inhibitor. In some embodiments, the CYP3A inhibitor is grapefruit juice, cobicistat, danoprevir, ritonavir, elvitegravir, indinavir, itraconazole, ketoconazole, lopinavir, paritaprevir, ombitasvir, dasabuvir, posaconazole, saquinavir, tipranavir, telithromycin, troleandomycin, voriconazole, aprepitant, ciprofloxacin, conivaptan, crizotinib, cyclosporine, diltiazem, dronedarone, erythromycin, fluconazole, fluvoxamine, imatinib, isavuconazole, tofisopam, verapamil, chlorzoxazone, cilostazol, cimetidine, clotrimazole, fosaprepitant, istradefylline, ivacaftor, lomitapide, ranitidine, ranolazine, or ticagrelor. In some embodiments, the CYP3A inhibitor is itraconazole.

A cytochrome P450 3A inducer, i.e., a “CYP3A inducer”, refers to a member of a class of compounds or a substance that increases activity of cytochrome P450 in humans. In some embodiments, the CYP3A inducer is a CYP3A4 inducer. In some embodiments, the CYP3A inducer is a barbiturate, corticosteroid, phenytoin, carbamazepine, rifampicin, or St. John's wort.

As used herein, the term “CDK inhibitor” refers to a compound that inhibits the enzymes in humans referred to as cyclin-dependent kinases (CDK). In some embodiments, the CDK inhibitor is a CDK4/6 inhibitor. As used herein, the term “CDK4/6 inhibitor” refers to a compound that inhibits CDK 4 and/or 6. Examples of a CDK inhibitor include, without limitation, SHR6390, trilaciclib, lerociclib, AT7519M, dinaciclib, ribociclib, abemaciclib, palbociclib, or any pharmaceutically acceptable salt thereof. In some embodiments, the CDK inhibitor is palbociclib or a pharmaceutically acceptable salt thereof.

As used herein, the term “PARP inhibitor” refers to a compound that inhibits the enzymes in humans referred to as poly ADP ribose polymerase (PARP). Examples of a PARP inhibitor include, without limitation, olaparib, rucaparib, talazoparib, niraparib, veliparib, pamiparib, CEP 9722, E7016, 3-aminobenzamide, mefuparib, and AZD2281.

As used herein, the term “anti-cancer agent” is used to describe an anti-cancer agent, or a therapeutic agent administered concurrently with an anti-cancer agent (e.g., palonosetron), with which may be co-administered and/or co-formulated with a compound of the disclosure (preferably Compound A or a pharmaceutically acceptable salt thereof) to treat cancer, and the side effects associated with the cancer treatment. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a CDK inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase (mek) inhibitor, a VEGF trap antibody, pemetrexed, erlotinib, dasatinib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Lonafarnib, BMS-214662, tipifarnib; amifostine, NVP-LAQ824, suberoyl anilide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, iodoxyfene, spironolactone, finasteride, cimetidine, trastuzumab, denileukin diftitox, gefitinib, bortezomib, paclitaxel, cremophor-free paclitaxel, docetaxel, epothilone B, BMS-247550, BMS-310705, droloxifene, 4-hydroxytamoxifen, pipendoxifene, ERA-923, arzoxifene, fulvestrant, aclonifen, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEGfilgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, and mixtures thereof. In some embodiments, the anti-cancer agent is selected from the group consisting of abiraterone, abiraterone acetate, estramustine, docetaxel, ketoconazole, goserelin, histrelin, triptorelin, buserelin, cyproterone, flutamide, bicalutamide, nilutamide, pamidronate, and zolendronate. In some embodiments, the anti-cancer agent is selected from the group consisting of FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, focal adhesion kinase inhibitor, Map kinase inhibitor, VEGF trap antibody, and chemical castration agent.

In some embodiments, the anti-cancer agent is selected from the group consisting of temozolomide, capecitabine, irinotecan, tamoxifen, anastrazole, exemestane, letrozole, DES, Estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, abiraterone acetate, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The terms “patient” and “subject” are used interchangeably herein, and refer to a mammal, e.g., a human, mouse, rat, guinea pig, dog, cat, horse, cow, pig, or non-human primate, such as a monkey, chimpanzee, baboon or rhesus.

In some embodiments, the subject is a human.

In some embodiments, the subject is a human who has been diagnosed with prostate cancer.

In some embodiments, the subject is a human who has been diagnosed with metastatic prostate cancer.

In some embodiments, the subject is a human who has been diagnosed with castrate-resistant prostate cancer.

In some embodiments, the subject is a human who has been diagnosed with metastatic castrate-resistant prostate cancer.

“Fasted condition” or “fasted state” as used to describe a subject herein means the subject has not eaten for at least 4 hours before a time point of interest, such as the time of administering Compound A. In an embodiment, a subject in the fasted state has not eaten for at least any of 6, 8, 10, or 12 hours prior to administration of Compound A or a pharmaceutically acceptable salt thereof.

“Fed condition” or “fed state” as used to describe a subject herein means the subject has eaten less than 4 hours before a time point of interest, such as the time of administering Compound A. In an embodiment, a subject in the fed state has eaten less than 3, 2, 1, or 0.5 hours prior to administration of Compound A or a pharmaceutically acceptable salt thereof.

### Methods of Ubiquitinating/Degrading a Target Protein in a Cell

The present disclosure provides a method of ubiquitinating/degrading a target protein in a cell. The method comprises administering a bifunctional composition comprising an E3 ubiquitin ligase binding moiety and a protein targeting moiety, preferably linked through a linker moiety, as otherwise described herein, wherein the E3 ubiquitin ligase binding moiety is coupled to the protein targeting moiety and wherein the E3 ubiquitin ligase binding moiety recognizes a ubiquitin pathway protein (e.g., an ubiquitin ligase, preferably an E3 ubiquitin ligase) and the protein targeting moiety recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

### Methods of Treatment

The methods of treating cancer described herein result in a reduction in tumor size. Alternatively, or in addition, the cancer is metastatic cancer, and this method of treatment includes inhibition of metastatic cancer cell invasion.

In some embodiments, the cancer is prostate cancer.

In some embodiments, the cancer is metastatic prostate cancer.

In some embodiments, the cancer is castrate-resistant prostate cancer.

In some embodiments, the cancer is metastatic castrate-resistant prostate cancer (mCRPC).

In one aspect, treating cancer results in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by 5% or greater relative to its volume prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In another aspect, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active agent or compound of the disclosure. In another preferred aspect, an increase in average survival time of a population may also be measured, for example, by calculating for a population the average length of survival following completion of a first round of treatment with an active agent or compound of the disclosure.

In another aspect, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured by calculating for a population the average length of survival following initiation of treatment with an active agent or compound of the disclosure. In another preferred aspect, an increase in average survival time of a population may be measured by calculating for a population the average length of survival following completion of a first round of treatment with a compound of the disclosure (preferably Compound A or a pharmaceutically acceptable salt thereof).

In another aspect, treating cancer results in a decrease in tumor growth rate. Preferably, after treatment, tumor growth rate is reduced by at least 5% relative to growth rate prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured by measuring an increase in the diameter of a tumor after a prior tumor shrinkage that followed treatment. In another preferred aspect, a decrease in tumor regrowth is indicated by failure of tumors to reoccur after treatment has stopped.

The dosages of Compound A, or the pharmaceutically acceptable salt thereof, for any of the methods and uses described herein vary depending on the agent, the age, weight, and clinical condition of the recipient subject, and the experience and judgment of the clinician or practitioner administering the therapy, among other factors affecting the selected dosage.

The therapeutically effective amount of Compound A, or the pharmaceutically acceptable salt thereof, may be administered one or more times over a day for up to 30 or more days, followed by 1 or more days of non-administration of the compound. This type of treatment schedule, i.e., administration of Compound A, or the pharmaceutically acceptable salt thereof, on consecutive days followed by non-administration of the compound on consecutive days may be referred to as a treatment cycle. A treatment cycle may be repeated as many times as necessary to achieve the intended affect.

In some embodiments, the therapeutically effective amount of Compound A is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg, or an equivalent amount of a pharmaceutically acceptable salt of Compound A, that is administered once, twice, three times, four times, or more daily for one, two, three, four, five, six, seven, eight, nine, ten, eleven, twelve, thirteen, fourteen, fifteen, twenty, twenty-five, thirty consecutive days, or, once, twice, three times, four times, or more daily, in single or divided doses, for 2 months, 3 months, 4 months, 5 months, 6 months, or longer.

In some embodiments, the therapeutically effective amount of Compound A is about 10 to about 40 mg, about 20 to about 50 mg, about 30 to about 60 mg, about 40 to about 70 mg, about 50 to about 80 mg, about 60 to about 90 mg, about 70 to about 100 mg, about 80 to about 110 mg, about 90 to about 120 mg, about 100 to about 130 mg, about 110 to about 140 mg, about 120 to about 150 mg, about 130 to about 160 mg, about 140 to about 170 mg, about 150 to about 180 mg, about 160 to about 190 mg, about 170 to about 200 mg, about 180 to about 210 mg, about 190 to about 220 mg, about 200 to about 230 mg, about 210 to about 240 mg, about 220 to about 250 mg, about 230 to about 260 mg, about 240 to about 270 mg, about 250 to about 280 mg, about 260 to about 290 mg, about 270 to about 300 mg, about 280 to about 310 mg, about 290 to about 320 mg, about 300 to about 330 mg, about 310 to about 340 mg, about 320 to about 350 mg, about 330 to about 360 mg, about 340 to about 370 mg, about 350 to about 380 mg, about 360 to about 390 mg, about 370 to about 400 mg, about 380 to about 410 mg, about 390 to about 420 mg, about 400 to about 430 mg, about 410 to about 440 mg, about 420 to about 450 mg, about 430 to about 460 mg, about 440 to about 470 mg, about 450 to about 480 mg, about 460 to about 490 mg, about 470 to about 500 mg, about 480 to about 510 mg, about 490 to about 520 mg, about 500 to about 530 mg, about 510 to about 540 mg, about 520 to about 550 mg, about 530 to about 560 mg, about 540 to about 570 mg, about 550 to about 580 mg, about 560 to about 590 mg, about 570 to about 600 mg, about 580 to about 610 mg, about 590 to about 620 mg, about 600 to about 630 mg, about 610 to about 640 mg, about 620 to about 650 mg, about 630 to about 660 mg, about 640 to about 670 mg, about 650 to about 680 mg, about 660 to about 690 mg, about 670 to about 700 mg, about 680 to about 710 mg, about 690 to about 720 mg, about 700 to about 730 mg, about 710 to about 740 mg, about 720 to about 750 mg, about 730 to about 760 mg, about 740 to about 770 mg, about 750 to about 780 mg, about 760 to about 790 mg, about 770 to about 800 mg, about 780 to about 810 mg, about 790 to about 820 mg, about 800 to about 830 mg, about 810 to about 840 mg, about 820 to about 850 mg, about 830 to about 860 mg, about 840 to about 870 mg, about 850 to about 880 mg, about 860 to about 890 mg, about 870 to about 900 mg, about 880 to about 910 mg, about 890 to about 920 mg, about 900 to about 930 mg, about 910 to about 940 mg, about 920 to about 950 mg, about 930 to about 960 mg, about 940 to about 970 mg, about 950 to about 980 mg, about 960 to about 990 mg, or about 970 to about 1,000 mg, or an equivalent amount of a pharmaceutically acceptable salt of Compound A, that is administered once, twice, three times, four times, or more daily in single or divided doses (which dose may be adjusted for the patient's weight in kg, body surface area in m2, and/or age in years).

In some embodiments, the therapeutically effective amount of Compound A is about 70 mg to about 1000 mg, or an equivalent amount of a pharmaceutically acceptable salt of Compound A, that is administered once, twice, three times, four times, or more daily in single or divided doses (which dose may be adjusted for the patient's weight in kg, body surface area in m2, and/or age in years).

In some embodiments, the therapeutically effective amount of Compound A is about 70 mg, 105 mg, 140 mg, 175 mg, 210 mg, 245 mg, 280 mg, 315 mg, 350 mg, 385 mg, 420 mg, 455 mg, 490 mg, 525 mg, 560 mg, 595 mg, 630 mg, 665 mg, or 700 mg, or an equivalent amount of a pharmaceutically acceptable salt of Compound A, that is administered once, twice, three times, four times, or more daily in single or divided doses (which dose may be adjusted for the patient's weight in kg, body surface area in m2, and/or age in years).

In some embodiments, the therapeutically effective amount of Compound A is about 100 mg, about 150 mg, or about 300 mg.

In some embodiments, the therapeutically effective amount of Compound A is about 100 mg.

In some embodiments, the therapeutically effective amount of Compound A is 100 mg.

In some embodiments, the therapeutically effective amount of Compound A is 100 mg, administered orally and once daily.

In some embodiments, the therapeutically effective amount of Compound A is about 150 mg.

In some embodiments, the therapeutically effective amount of Compound A is 150 mg.

In some embodiments, the therapeutically effective amount of Compound A is 150 mg, administered orally and once daily.

In some embodiments, the therapeutically effective amount of Compound A is about 300 mg.

In some embodiments, the therapeutically effective amount of Compound A is 300 mg.

In some embodiments, the therapeutically effective amount of Compound A is 300 mg, administered orally and once daily.

The therapeutically effective amount of Compound A can also range from about 0.01 mg/kg per day to about 100 mg/kg per day. In an aspect, therapeutically effective amount of Compound A can range from about 0.05 mg/kg per day to about 10 mg/kg per day. In an aspect, therapeutically effective amount of Compound A can range from about 0.075 mg/kg per day to about 5 mg/kg per day. In an aspect, therapeutically effective amount of Compound A can range from about 0.10 mg/kg per day to about 1 mg/kg per day. In an aspect, therapeutically effective amount of Compound A can range from about 0.20 mg/kg per day to about 0.70 mg/kg per day.

In some embodiments, the therapeutically effective amount of Compound A is about 0.10 mg/kg per day, about 0.15 mg/kg per day, about 0.20 mg/kg per day, about 0.25 mg/kg per day, about 0.30 mg/kg per day, about 0.35 mg/kg per day, about 0.40 mg/kg per day, about 0.45 mg/kg per day, about 0.50 mg/kg per day, about 0.55 mg/kg per day, about 0.60 mg/kg per day, about 0.65 mg/kg per day, about 0.70 mg/kg per day, about 0.75 mg/kg per day, about 0.80 mg/kg per day, about 0.85 mg/kg per day, about 0.90 mg/kg per day, about 0.95 mg/kg per day, or about 1.00 mg/kg per day.

In some embodiments, the therapeutically effective amount of Compound A is about 1.05 mg/kg per day, about 1.10 mg/kg per day, about 1.15 mg/kg per day, about 1.20 mg/kg per day, about 1.25 mg/kg per day, about 1.30 mg/kg per day, about 1.35 mg/kg per day, about 1.40 mg/kg per day, about 1.45 mg/kg per day, about 1.50 mg/kg per day, about 1.55 mg/kg per day, about 1.60 mg/kg per day, about 1.65 mg/kg per day, about 1.70 mg/kg per day, about 1.75 mg/kg per day, about 1.80 mg/kg per day, about 1.85 mg/kg per day, about 1.90 mg/kg per day, about 1.95 mg/kg per day, or about 2.00 mg/kg per day.

In some embodiments, the therapeutically effective amount of Compound A is about 2 mg/kg per day, about 2.5 mg/kg per day, about 3 mg/kg per day, about 3.5 mg/kg per day, about 4 mg/kg per day, about 4.5 mg/kg per day, about 5 mg/kg per day, about 5.5 mg/kg per day, about 6 mg/kg per day, about 6.5 mg/kg per day, about 7 mg/kg per day, about 7.5 mg/kg per day, about 8.0 mg/kg per day, about 8.5 mg/kg per day, about 9.0 mg/kg per day, about 9.5 mg/kg per day, or about 10 mg/kg per day.

In some embodiments, the therapeutically effective amount of Compound A, or the pharmaceutically acceptable salt thereof, is administered to the subject once daily. In some embodiments, this daily dose of Compound A, or the pharmaceutically acceptable salt thereof, may be administered to the subject all at once. In some embodiments, this daily dose of Compound A, or the pharmaceutically acceptable salt thereof, may be administered to the subject in two portions (i.e., a divided dose). In some embodiments, this daily dose of Compound A, or the pharmaceutically acceptable salt thereof, may be administered to the subject in three divided doses. In some embodiments, this daily dose of Compound A, or the pharmaceutically acceptable salt thereof, may be administered to the subject in four divided doses. In some embodiments, this daily dose of Compound A, or the pharmaceutically acceptable salt thereof, may be administered to the subject in five or more divided doses. In some embodiments, these portions or divided doses are administered to the subject at regular intervals throughout the day, for example, every 12 hours, every 8 hours, every 6 hours, every 5 hours, every 4 hours, etc.

The therapeutically effective amount of Compound A, or the pharmaceutically acceptable salt thereof, can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of Compound A or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, once every two weeks, or monthly depending on half-life and clearance rate of the particular formulation.

In some embodiments, for the methods of treating prostate cancer with the combination of Compound A, or a pharmaceutically acceptable salt thereof, and another anti-cancer agent, the therapeutically effective amount of Compound A, or a pharmaceutically acceptable salt thereof, is described herein, and the therapeutically effective amount of the anti-cancer agent is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, or thirty consecutive days, or, once, twice, three times, four times, or more daily, in single or divided doses, for 2 months, 3 months, 4 months, 5 months, 6 months, or longer.

### Pharmaceutical Compositions

In some embodiments, Compound A, or the pharmaceutically acceptable salt thereof, is formulated for oral administration. For example, in some embodiments, the compound of the disclosure is formulated as a tablet that comprises zero, one, two, or more of each of the following: emulsifier; surfactant, binder; disintegrant, glidant; and lubricant.

In some embodiments, the emulsifier is hypromellose.

In some embodiments, the surfactant is vitamin E polyethylene glycol succinate.

In some embodiments, the binder (also referred to herein as a filler) is selected from the group consisting of microcrystalline cellulose, lactose monohydrate, sucrose, glucose, and sorbitol.

In some embodiments, the disintegrant is croscarmellose sodium.

In some embodiments, the glidant refers to a substance used to promote powder flow by reducing interparticle cohesion. In some embodiments, in the dosage forms of the disclosure, the glidant is selected from the group consisting of silicon dioxide, silica colloidal anhydrous, starch, and talc.

In some embodiments, the lubricant refers to a substance that prevents ingredients from sticking and/or clumping together in the machines used in preparation of the dosage forms of the disclosure. In some embodiments, in the dosage forms of the disclosure, the lubricant is selected from the group consisting of magnesium stearate, sodium stearyl fumarate, stearic acid, and vegetable stearin.

The pharmaceutical compositions containing Compound A, or the pharmaceutically acceptable salt thereof, may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of Compound A, or the pharmaceutically acceptable salt thereof, into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating Compound A, or the pharmaceutically acceptable salt thereof, in the required amount in an appropriate solvent with one or a combination of ingredients enumerated above, as required, followed by filtered sterilization. Generally, dispersions are prepared by incorporating the active agent or compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets.

For the purpose of oral therapeutic administration, Compound A, or the pharmaceutically acceptable salt thereof, can be incorporated with excipients and used in the form of tablets, troches, or capsules. Oral compositions can also be prepared using a fluid carrier for use as a mouthwash, wherein the agent or compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the agents or compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents or compounds are formulated into ointments, salves, gels, or creams as generally known in the art.

In one aspect, Compound A, or the pharmaceutically acceptable salt thereof, is prepared with pharmaceutically acceptable carriers that will protect the agent or compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent or compound calculated to produce the desired therapeutic effect in association with the required pharmaceutical carrier. The specification for the dosage unit forms of the application is dictated by and directly dependent on the unique characteristics of Compound A, or the pharmaceutically acceptable salt thereof, and the particular therapeutic effect to be achieved.

The pharmaceutical compositions can be included in a container, pack, or dispenser together with instructions for administration.

Illustrative modes of administration for Compound A, or the pharmaceutically acceptable salt thereof, includes systemic or local administration such as oral, nasal, parenteral, transdermal, subcutaneous, vaginal, buccal, rectal or topical administration modes. In some embodiments, Compound A, or the pharmaceutically acceptable salt thereof, is administered orally. In some embodiments, Compound A, or the pharmaceutically acceptable salt thereof, is administered as a tablet, capsule, caplet, solution, suspension, syrup, granule, bead, powder, or pellet.

Illustrative pharmaceutical compositions are tablets and gelatin capsules comprising a salt of Compound A and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the salt such as cyclodextrin, hydroxypropyl-cyclodextrin, PEG400, and/or PEG200.

For preparing pharmaceutical compositions from Compound A, or a salt or hydrate thereof, inert, pharmaceutically acceptable carriers can be either solid or liquid. Solid form preparations include powders, tablets, dispersible granules, capsules, cachets and suppositories.

The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed salt is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions and emulsions.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, intrathecal, subcutaneous or intramuscular form, and all using forms well known to those skilled in the pharmaceutical arts.

Pharmaceutical compositions can be prepared according to conventional mixing, granulating or coating methods, respectively, and the present pharmaceutical compositions can contain from about 0.1% to about 99%, from about 5% to about 90%, or from about 1% to about 20% of the disclosed free base or salt by weight or volume.

The pharmaceutical compositions containing Compound A or the pharmaceutically acceptable salt thereof, may further comprising one or more additional anti-cancer agents, including any of those disclosed herein.

All amounts of any component of an oral dosage form described herein, e.g., a tablet, which is indicated based on % w/w, refers to the total weight of the oral dosage form, unless otherwise indicated.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1—In Vitro Evaluation of Compound A for Cytochrome P450- and Transporter-Mediated Drug-Drug Interaction

In vitro studies were conducted to assess the potential of Compound A to cause cytochrome P450 (CYP) and transporter-mediated drug-drug interactions (DDI).

The induction potential of Compound A on CYP enzymes was assessed in cryopreserved human hepatocytes from three donors. Following treatment for with Compound A at concentrations of 0.03-30 μM for 48 hours, mRNA levels for CYP1A2, 2B6, 2C8, 2C9, 2C19, and 3A4 were determined by semiquantitative real-time polymerase chain reaction (PCR). Test article concentrations during 24-hour incubation were determined. The potential of Compound A to cause direct and time-dependent inhibition (TDI) of the activities of CYP1A2, 2B6, 2C8, 2C9, 2C19, 2D6, and 3A4 was evaluated in pooled human liver microsomes (HLM) at 0.2-15 μM. Probe substrate concentrations around Km were used for direction inhibition and initial TDI assays. For TDI assay, Compound A was pre-incubated with pooled HLM with and without NADPH for 30 minutes prior to incubation with a single concentration of probe substrate.

Metabolite profiling was conducted in incubations of Compound A (2 and 10 μM) with HLM (up to 60 min), human hepatocyte suspension (up to 240 min) and human plasma (up to 360 min). In addition, Compound A (2 μM) was incubated with recombinant human CYP enzymes CYP1A2, CYP2A6, CYP2B6, CYP2C8, CYP2C9, CYP2C19, CYP2D6, CYP2E1, CYP3A4 and CYP3A5.

The DDI potential of Compound A as a victim via CYP mediated pathways was examined in HLM, human hepatocyte suspensions, and human recombinant CYP enzymes.

In addition, the inhibition potential against efflux transporters (P-glycoprotein (Pgp) and breast cancer resistance protein (BCRP)) and uptake transporters (MATE1, MATE2-K, OATP1B1, OATP1B3, OAT1, OAT3, and OCT2) was assessed using single transporter over-expressed cell monolayers (MDCK II or HEK293) or inside-out vesicles.

The potential of Compound A to inhibit Pgp or BCRP was tested for bidirectional transport of the probe substrates in Pgp or BCRP-expressed MDCKII and control monolayers at Compound A concentrations of 0.07-5 μM. Pgp and BCR inhibition were tested in inside-out membrane vesicles prepared from HEK293 overexpressing human Pgp and BCR at concentrations of 0.01-9 μM Compound A in the presence of 4 mM MgATP or MgAMP. The potential of Compound A to inhibit uptake transporter in MDCKII or HEK293 cells stable expressing single MATE1, MATE2-K, OAT1, OAT3, OATP1B1, OATP1B3 and OCT2 in was tested at concentrations of 0.005-3.75 μM.

The potential of Compound A to act as an efflux transporter Pgp and BCRP as well as uptake transporter OATP1B1 and 1B3 were assessed in Caco-2 and single transporter over-expressed HEK29 cells, respectively.

The potential of Compound A to act as a substrate for Pgp and BCRP was examined in Caco-2 cells at the concentrations of 0.075, 0.75, 3.75 and 7.5 μM containing 1% of BSA. After 120 min incubation, the bidirectional permeability of Compound A was determined by LC-MS/MS. The involvement of Compound A as a substrate of OATP1B1 and OATP1B3 was assessed in the uptake transporter substrate assays at 4 concentrations (0.1, 0.5, 1 and 5 μM). Compound A in cell lysates was determined by LC-MS/MS.

### Results

CYP Induction: no significant decrease of hepatocyte viability was found in hepatocytes at all Compound A concentrations (0.03-30 μM) after 2 days treatment in the upfront MTT assay (data not shown). Positive control inducers behaved as expected (Table 1). Maximal 2.5-8.8-fold induction (4-6% of positive control response) in CYP3A4 mRNA were found at 0.1-0.3 μM in all three donors, and 3.1-fold for CYP2C8 mRNA in one of three lots (donor 3 only) at 0.1 μM (28% of positive control response). The induction response was attenuated at higher concentrations (Table 1).

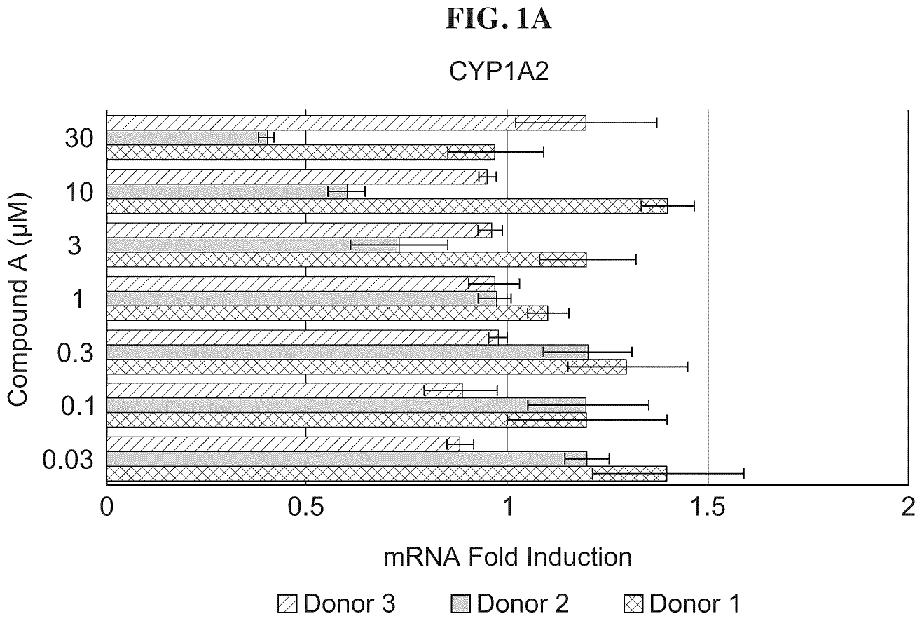

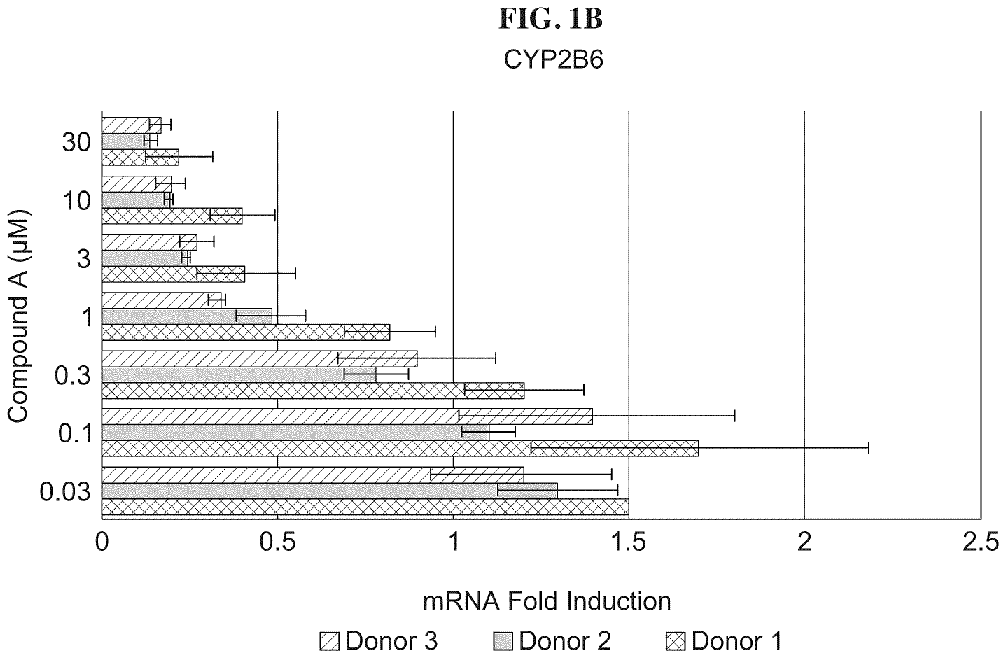

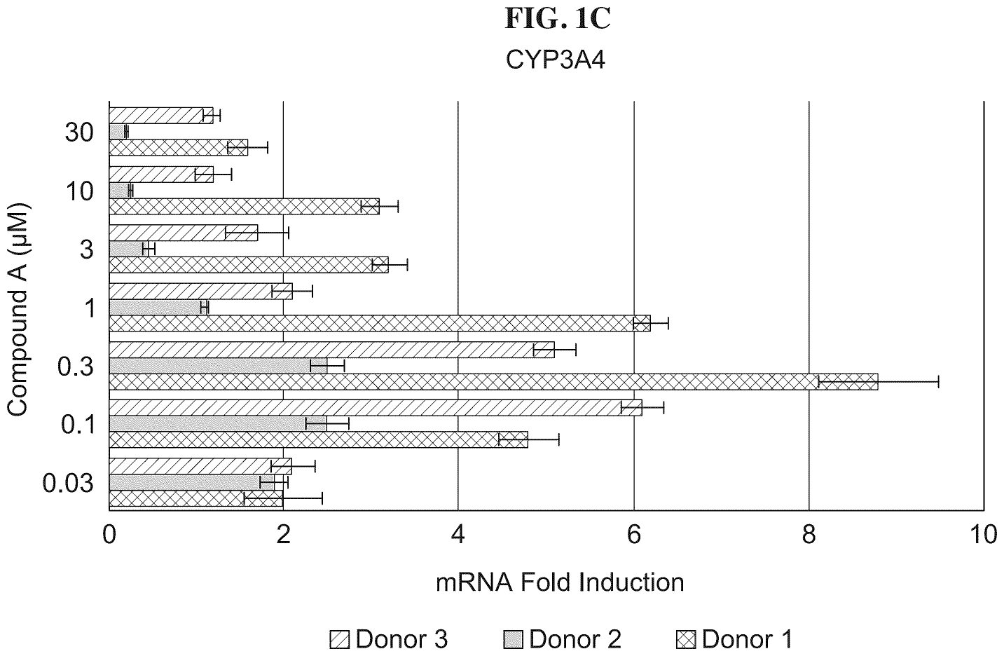

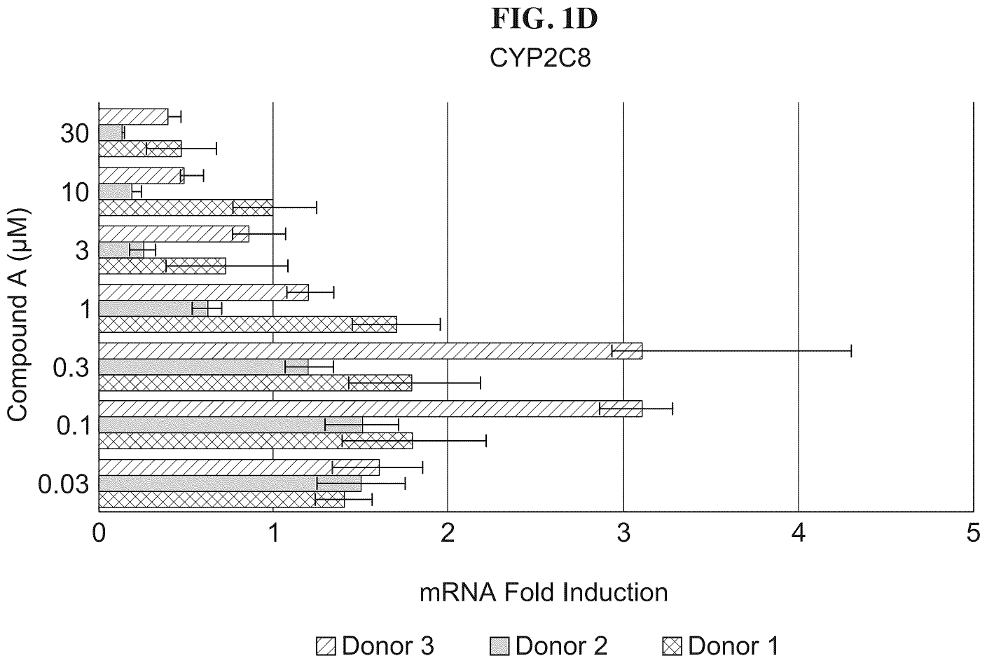

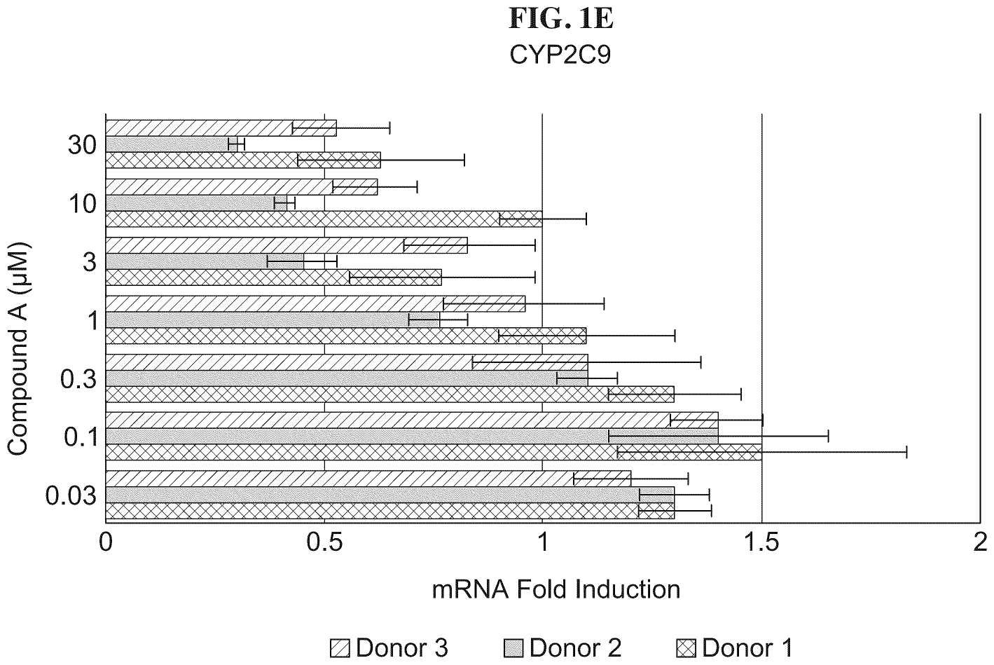

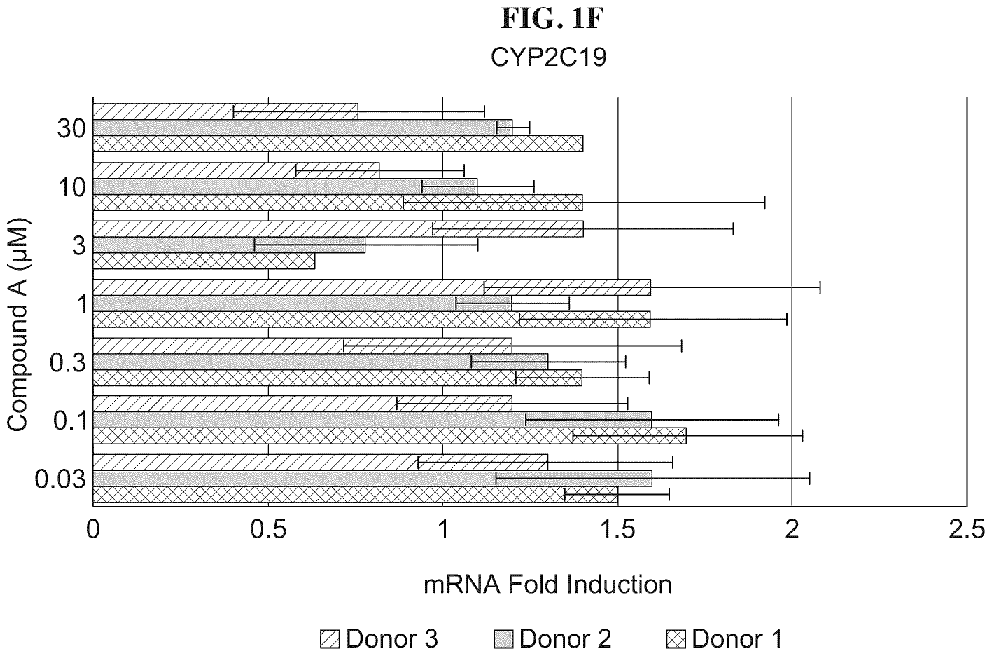

At concentrations ranging from 0.03-30 μM, Compound A did not induce mRNA of CYP1A2, 2B6, and 2C9 for all three donors of human hepatocytes (Table 1 and FIG. 1).

TABLE 1
Effect of Compound A on CYP mRNA
Expression in Human Hepatocytes

| | Donor 1 | | Donor 2 | | Donor 3 | | PC* |
|---|---|---|---|---|---|---|---|
| Enzyme | Fold | % PC | Fold | % PC | Fold | % PC | Fold |
| CYP1A2 | <2 | — | <2 | — | <2 | — | 77-88 |
| CYP2B6 | <2 | — | <2 | — | <2 | — | 14-25 |
| CYP2C8 | <2 | — | <2 | — | 3.1 | 28 | 1.6-8.6 |
| CYP2C9 | <2 | — | <2 | — | <2 | — | 2.0-3.8 |
| CYP2C19 | <2 | — | <2 | — | <2 | — | 0.93-1.4 |
| CYP3A4 | 8.8 | 5 | 2.5 | 6 | 3.3 | 5 | 26-149 |

*PC: positive control inducer, Omeprazole for CYP1A2, Phenobarbital for CYP2B6, Rifampicin for CYP2Cs and 3A4)

CYP Inhibition: no direct or time-dependent inhibition was observed for any of the CYP isoforms after incubating HLM with Compound A at concentrations of 0.2-15 UM (Table 2). Positive control inhibitors demonstrated direct and TDI for all enzymes tested, with expected IC₅₀ values and fold shift (data not shown). Compound A did not cause direct inhibition (≤12% maximal inhibition) and TDI for all CYPs (≤15%) up to 15 UM tested (Table 2).

TABLE 2
Effect of Compound A on Direct and Time-dependent
Inhibition in Pooled Human Liver Microsomes

| | | IC₅₀ Values (μM) | | |
|---|---|---|---|---|
| | Substrate | No Pre- | 30 min Pre- | IC₅₀ Shift |
| Enzyme | μM | incubation | incubation | Fold |
| CYP1A2 | Phenacetin | >15 | >15 | NA* |
| | 50 | | | |
| CYP2B6 | Bupropion | >15 | >15 | NA |
| | 50 | | | |
| CYP2C8 | Amodiaquine | >15 | >15 | NA |
| | 2 | | | |
| CYP2C9 | Diclofenac | >15 | >15 | NA |
| | 5 | | | |
| CYP2C19 | S-Mephenytoin | >15 | >15 | NA |
| | 20 | | | |
| CYP2D6 | Bufuralol | >15 | >15 | NA |
| | 5 | | | |
| CYP3A | Midazolam | >15 | >15 | NA |
| | 2 | | | |
| CYP3A | Testosterone | >15 | >15 | NA |
| | 50 | | | |

*NA: Not applicable

Metabolism and CYP Reaction Phenotyping: Compound A was relatively stable in HLM incubations up to 60 min at 2 μM, human hepatocyte suspension (up to 240 min) and major recombinant CYPs (up to 25 min) (data not shown). Up to a 23% loss of Compound A was observed in CYP3A5 (data not shown).

Metabolite profiling using human plasma, HLM and human hepatocytes indicated that hydrolysis (M825/1) was the major metabolic pathway. Other minor pathways included oxidation, de-alkylation, demethylation, which represent <2% of total abundance (Table 3).

TABLE 3
Metabolite Profile of Compound A in Human Liver Microsomes (HLM),
Human Hepatocyte (HH) Suspensions and Human Plasma (HP)

| Metabolite | | % Parent at 0 Min | | |
|---|---|---|---|---|
| Code | Reaction | HLM at 60 min | HH at 240 min | HP at 360 min |
| Compound A | N.A. | 92.6 | 86.9 | 77.0 |
| (Parent) | | | | |
| M334/1 | De-alkylation | 0.24 | 0.28 | — |
| M505/1 | De-alkylation + Oxidation | 0.65 | 1.23 | — |
| M509/1 | De-alkylation + Oxidation + | 0.04 | — | — |
| | Reduction + De-methylation | | | |
| M521/1 | De-alkylation + Oxidation | 0.02 | — | — |
| M793/1 | De-methylation | 0.05 | — | — |
| M823/1 | Oxidation | 0.31 | 0.52 | — |
| M825/1 | Hydrolysis | — | — | 42.3 |

Efflux Transporter Substrate and Inhibition: Compound A exhibited low permeability in Caco-2 cell monolayers (data not shown). Due to the low permeability values, Compound A as a substrate for Pgp and BCRP was not reliably determined.

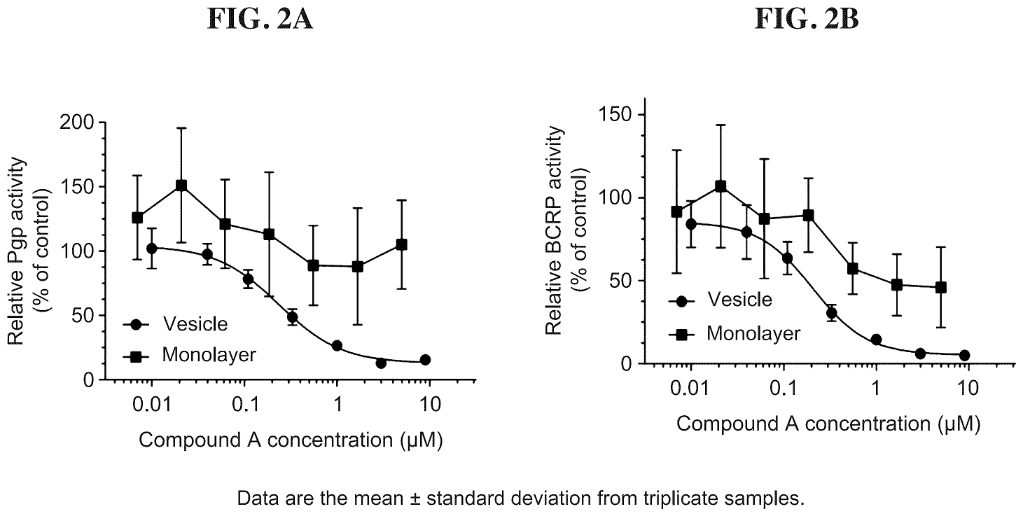

Compound A inhibited Pgp in vesicle assays with an IC₅₀ value of 0.23 UM but the inhibition was not observed in the MDCKII bidirectional assays. Compound A inhibited BCRP in both monolayer and vesicle assays with IC₅₀ values of 1.55 UM and 0.21 μM, respectively (Table 4 and FIG. 2). Positive control probe substrates and inhibitors demonstrated functional assay systems (data not shown).

TABLE 4
Pgp and BCRP Inhibition by Compound A

| | | | IC₅₀ Values (μM) | |
|---|---|---|---|---|
| | | | Maximal % | IC50 |
| Transporter | Assay Type | Substrate (μM) | Inhibition | Values (μM) |
| Pgp | Monolayers | Digoxin (5) | <20 | >5 |
| | Vesicles | NMQ* (1) | 87 | 0.23 |
| BCRP | Monolayers | Prazosin (1) | 54 | 1.55 |
| | Vesicles | Rosuvastatin | 95 | 0.21 |
| | | (1) | | |

*NMQ: N-methyl-quinidine

Efflux Transporter Substrate and Inhibition: probe substrates and inhibitors demonstrated expected uptake activity and inhibition for each transporter (data not shown). Compound A was not a substrate for OATP1B1 and OATP1B3 as the accumulation of Compound A was similar in the OATP1B1/1B3-expressing and control cell (fold accumulations <2), indicating no active accumulation of Compound A under the conditions tested (data not shown).

Compound A did not cause >50% inhibition for the following transporters: OATP1B1, OATP1B3, OAT1, OAT3, OCT2, and MATE2-K. However, at a concentration of 3.75 μM, Compound A resulted in a 52% inhibition of MATE1 (EC₅₀: 3.05 μM. Tests with higher concentrations were limited by solubility. (Table 5).

TABLE 5
Uptake Transporter Inhibition by Compound A

| | | IC₅₀ Values (μM) | |
|---|---|---|---|
| | | Maximal % | IC50 |
| Transporter | Substrate (μM) | Inhibition | Values (μM) |
| OATP1B1 | Rosuvastatin (1) | 37 | >3.75 |
| OATP1B3 | Rosuvastatin (1) | 22 | >3.75 |
| OAT1 | Estrone-3-sulfate (E3S) (5) | 19 | >3.75 |
| OAT3 | E3S* (1) | 13 | >3.75 |
| OCT2 | Metformin (10) | 28 | >3.75 |
| MATE1 | Metformin (10) | 52 | 3.05 |
| MATE2-K | Metformin (10) | 44 | >3.75 |

Without wishing to be bound by theory, these results seem to demonstrate that Compound A has a low potential to cause significant DDI as an inhibitor or substrate of CYP enzymes. Moreover, the results also seem to demonstrate that Compound A has a low potential to cause DDI of uptake transporters.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

The methods of the disclosure have been described herein by reference to certain preferred embodiments. However, as particular variations thereon will become apparent to those skilled in the art, based on the disclosure set forth herein, the disclosure is not to be considered as limited thereto.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise.

It is to be understood that at least some of the descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

Further, to the extent that a method does not rely on the particular order of steps set forth herein, the particular order of the steps recited in a claim should not be construed as a limitation on that claim.

All patents, patent applications, references and publications cited herein are fully and completely incorporated by reference as if set forth in their entirety. Such documents are not admitted to be prior art to the present disclosure.

1. A method of treating prostate cancer in a subject in need thereof, comprising
administering to the subject a therapeutically effective amount of Compound A, 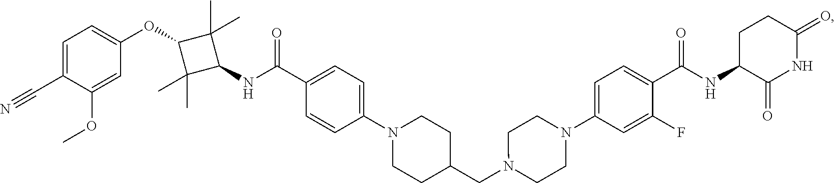 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof, and
discontinuing or reducing the administration of a CYP3A inhibitor or inducer, an efflux transporter substrate or inhibitor, or an uptake transporter substrate or inhibitor, to the subject prior to initiating administration of Compound A, or the pharmaceutically acceptable salt thereof.

2. A method of treating prostate cancer in a subject in need thereof, comprising
administering to the subject a therapeutically effective amount of Compound A, 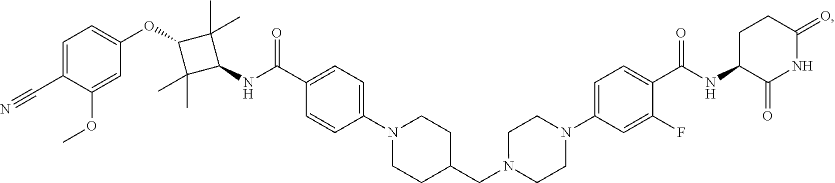 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
and
discontinuing or reducing the administration of a CYP3A inhibitor or inducer, an efflux transporter substrate or inhibitor, or an uptake transporter substrate or inhibitor, to the subject prior to initiating administration of Compound A.

3. The method of claim 1, further comprising discontinuing the administration of the CYP3A inhibitor or inducer, efflux transporter substrate or inhibitor, or uptake transporter substrate or inhibitor, to the subject prior to initiating administration of Compound A, or the pharmaceutically acceptable salt thereof.

4. The method of claim 1, further comprising reducing the administration of the CYP3A inhibitor or inducer, efflux transporter substrate or inhibitor, or uptake transporter substrate or inhibitor, to the subject prior to initiating administration of Compound A, or the pharmaceutically acceptable salt thereof.

5. The method of claim 3, wherein the administration of the CYP3A inhibitor or inducer, efflux transporter substrate or inhibitor, or uptake transporter substrate or inhibitor is discontinued in the subject beginning at a time point prior to initiating the administration of Compound A, or the pharmaceutically acceptable salt thereof, wherein the time point is at least 120 hours.

6. The method of claim 4, wherein the administration of the CYP3A inhibitor or inducer, efflux transporter substrate or inhibitor, or uptake transporter substrate or inhibitor is reduced in the subject at a time point prior to initiating the administration of Compound A, or the pharmaceutically acceptable salt thereof, wherein the time point is at least 120 hours.

7. A method of treating prostate cancer in a subject in need thereof, comprising
administering to the subject a therapeutically effective amount of Compound A, 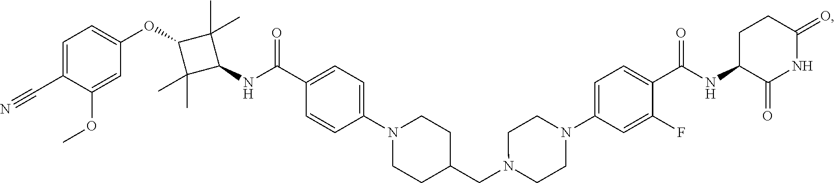 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof, and
administering a CYP3A inhibitor or inducer, an efflux transporter substrate or inhibitor, or an uptake transporter substrate or inhibitor, to the subject.

8. (canceled)

9. The method of claim 1, wherein the prostate cancer is castrate-resistant prostate cancer.

10. The method of claim 1, wherein the prostate cancer is metastatic prostate cancer.

11. The method of claim 1, wherein the prostate cancer is metastatic castrate-resistant prostate cancer.

12. The method of claim 1, wherein the subject is in a fed state.

13. The method of claim 1, wherein the subject is in a fasted state.

14. The method of claim 1, wherein the efflux transporter is Pgp or BCRP.

15. The method of claim 1, wherein the uptake transporter is OATP1B1, OATP1B3, OAT1, OAT3, OCT2, MATE1, or MATE2-K.
